(19) United States
(12) Patent Application Publication
(10) Pub. No.: US 2024/0228414 A9
(43) Pub. Date: Jul. 11, 2024

(54) Conversion of carbon dioxide and water to synthesis gas for producing methanol and hydrocarbon products

(71) Applicant: TOPSOE A/S, Lyngby (DK)

(72) Inventors: Christian WIX, Naerum (DK); Troels STUMMANN, Copenhagen (DK)

(73) Assignee: TOPSOE A/S, Kgs. Lyngby (DK)

(21) Appl. No.: 18/256,689

(22) PCT Filed: Dec. 21, 2021
(86) PCT No.: PCT/EP2021/086999

§ 371 Date: Jun. 9, 2023

(30) Foreign Application Priority Data
Dec. 22, 2020 (EP) 20216617.9

(51) Int. Cl. C07C 29/151; C07C 29/80; C10G 2/00; C25B 1/042; C25B 1/23; C25B 9/23; C25B 15/08

(52) U.S. Cl. CPC C07C 29/1518; C07C 29/80; C10G 2/30; C25B 1/042; C25B 1/23; C25B 9/23; C25B 15/081

(57) ABSTRACT

A method and system for producing a synthesis gas for use in the production of methanol, or a hydrocarbon product such as a synthetic fuel, comprising the steps of: providing a carbon dioxide-rich stream and passing it through an electrolysis unit for producing a feed stream comprising CO and CO₂; providing a water feedstock and passing it through an electrolysis unit for producing a feed stream comprising H₂; combining said feed stream comprising CO and CO₂ and said feed stream comprising H₂ into said synthesis gas; and converting said synthesis gas into said methanol or said hydrocarbon product.

### FIELD OF THE INVENTION

The invention relates to a method and a system for producing a synthesis gas from a carbon dioxide-rich stream and a water feedstock, where the synthesis gas is used for the production of methanol by methanol synthesis, or a hydrocarbon product, in particular a synthetic fuel such as diesel, by Fischer-Tropsch synthesis (FT).

### BACKGROUND OF THE INVENTION

Currently it is often inefficient and problematic to produce methanol and FT-hydrocarbon products from H₂ and CO₂, e.g. from a synthesis gas, this being a gas rich in H₂ and CO₂ and normally produced by steam reforming of a hydrocarbon feedstock such as natural gas. For methanol synthesis, a high CO₂ to CO ratio in the synthesis gas results in a larger methanol conversion reactor and more expensive downstream purification process. For FT, some of the CO₂ will have to be converted to CO using the reverse water gas shift reaction (water gas shift reaction, WGS: CO+H₂O=CO₂+H₂). This represents an expensive and complex solution involving i.a. the use of shift converters for conducting the reverse WGS reaction.

For methanol production purposes, it is known to use electrolysis of water to produce H₂ and then mix it with CO₂ to form a synthesis gas. For FT there is no standard solution, with the use of reverse WGS so far being the most viable solution, and yet nothing commercially has been built.

Hence, a known way of producing methanol is by taking a water feedstock and via electrolysis converting it into H₂, and then combining with a separate CO₂-rich stream and compressing for thereby forming a synthesis gas having a molar ratio H₂/CO₂ of about 3. This synthesis gas is then passed to a conventional methanol loop including conversion into methanol (CH₃OH) in a methanol synthesis reactor according to the reactions: 3 H₂+CO₂═CH₃OH+H₂O, CO+2 H₂═CH₃OH. The resulting raw methanol stream is then purified, i.e. enriched in methanol, via distillation, thereby producing a product stream with at least 98 wt % methanol as well as a separate water stream.

Applicant's WO 20208008 A1 discloses a plant, such as a hydrocarbon plant, which consists of a syngas (synthesis gas) stage comprising autothermal reforming for syngas generation and a synthesis stage where said syngas is synthesized to produce syngas derived product, such as hydrocarbon product or methanol. The plant makes effective use of various streams; in particular CO₂ and H₂. The plant does not comprise an external feed of hydrocarbons.

US 2007045125 A1 discloses a method for synthesizing synthesis gas from carbon dioxide obtained from atmospheric air or other available carbon dioxide source and water using a sodium-conducting electrochemical cell. Synthesis gas is also produced by the co-electrolysis of carbon dioxide and steam in solid oxide electrolytic cell. The synthesis gas produced may then be further processed and eventually converted into a liquid fuel suitable for transportation or other applications. This citation is at least silent on the use of a solid oxide electrolysis unit for conversion of CO₂ to a specific mixture of CO and CO₂.

US 20090289227 A1 discloses a method for utilizing CO₂ waste comprising recovering carbon dioxide from an industrial process that produces a waste stream comprising carbon dioxide in an amount greater than an amount of carbon dioxide present in starting materials for the industrial process. The method further includes producing hydrogen using a renewable energy resource and producing a hydrocarbon material utilizing the produced hydrogen and the recovered carbon dioxide. The carbon dioxide may be converted to CO by electrolysis and water to hydrogen by electrolysis. This citation is at least silent on the use of a solid oxide electrolysis unit for conversion of CO₂ to a specific mixture of CO and CO₂.

US 20180127668 A1 discloses a renewable fuel production system includes a carbon dioxide capture unit for extracting carbon dioxide from atmospheric air, a carbon dioxide electrolyzer for converting carbon dioxide to carbon monoxide, a water electrolyzer for converting water to hydrogen, a synfuels generator for converting carbon monoxide produced by the carbon dioxide electrolyzer and hydrogen produced by the water electrolyzer to a fuel. The fuel produced can be synthetic gasoline and/or synthetic diesel. The carbon dioxide is converted to CO via an electrochemical conversion of CO₂, which refers to any electrochemical process in which carbon dioxide, carbonate, or bicarbonate is converted into another chemical substance in any step of the process. This citation is therefore at least silent on the use of a solid oxide electrolysis unit for conversion of CO₂, as well as converting the CO₂ to a specific mixture of CO and CO₂.

### SUMMARY OF THE INVENTION

It has now been found that by using a combination of electrolysis steps for both a water feed and a CO₂ feed, it is now possible to form a more reactive synthesis gas for subsequent methanol conversion and/or for production of hydrocarbon products such as synthetic fuels, resulting i.a. in reduction of reactor size such as size of a methanol converter, less formation of water and not least a drastic reduction of the carbon foot-print. Furthermore, savings in terms of hydrogen consumption for particularly methanol conversion are achieved as well. Other associated benefits will become apparent from the below embodiments.

Accordingly, in a first aspect the invention is a method for producing methanol, comprising the steps of:
providing a carbon dioxide-rich stream and passing it through an electrolysis unit for producing a feed stream comprising CO and CO₂,
providing a water feedstock and passing it through an electrolysis unit for producing a feed stream comprising H₂,
combining said feed stream comprising CO and CO₂ and said feed stream comprising H₂ into a synthesis gas,
converting said synthesis gas into said methanol,
   wherein the step of providing a carbon dioxide-rich stream and passing it through an electrolysis unit for producing a feed stream comprising CO and CO₂, is conducted as a once-through operation in a solid oxide electrolysis cell unit i.e. SOEC-CO₂, and
   wherein the molar ratio CO/CO₂ in the feed stream comprising CO and CO₂, or the synthesis gas, is in the range 0.2-0.6, such as 0.25 or 0.30 or 0.35, 0.40 or 0.45, 0.50 or 0.55.

As used herein, the term “passing it through” means that electrolysis process is occurring in the electrolysis unit, whereby at least part of e.g. the carbon dioxide is converted into CO with the help of electric current.

By the invention, the feed stream comprising CO and CO₂, or the synthesis gas has a molar ratio CO/CO₂ in the range 0.2-0.6, such as 0.25 or 0.30 or 0.35, 0.40 or 0.45, 0.50 or 0.55. A synthesis gas having a CO/CO₂ in this range, particularly a molar ratio of e.g. 0.55 (i.e. about 65:35 CO₂:CO, approximately corresponding to a molar ratio CO₂/CO of 1.82), is much more reactive than one based on pure CO₂. The cost and the energy consumption of the methanol plant is therefore reduced when using the thus partly converted CO₂ stream. By operating with a molar ratio CO/CO₂ higher than 0.6 or higher, there is a risk of carbon formation due to the higher content of CO in the gas, while operation at a molar ratio CO/CO₂ below 0.2 is inexpedient, as i.a. the associated capital expense of the electrolysis unit per converted produced CO molecule becomes too high.

The feed stream comprising CO and CO₂, or the synthesis gas, has a molar ratio CO/CO₂ of 0.2 or higher, as recited above, thus enables a partial conversion being conducted. The electrolysis is thereby purposely conducted so that more CO is produced and the resulting molar ratio of CO to CO₂ is 0.2 or above 0.2, such as above 0.3 or above 0.4 or 0.5, for instance 0.6, thereby enabling easier tailoring of the relative content of CO, CO₂ and H₂ in the resulting synthesis gas to the proper module as it is described below for subsequent conversion to methanol when molar ratio of CO to CO₂ is 0.2-0.6, or to the proper H₂/CO molar ratio for conversion to hydrocarbon products when the molar ratio of CO to CO₂ is 0.8 or higher such as 0.9, as it is also described in more detail in a below separate embodiment. In this embodiment, the molar ratio CO/CO₂ is 0.8 or higher such as 0.9, or even higher, enables a much more suitable synthesis gas for downstream conversion of the synthesis gas into the hydrocarbon product, where it is desirable to have as much CO as possible in the gas compared to CO₂. For instance, the amount of hydrogen formed from the electrolysis of the water feedstock is normally too high to ensure the module or H₂/CO molar ratio reaching a value in the desired range, thus forcing the use of a portion of the hydrogen for other purposes. In other words, if too much H₂ is produced, the H₂/CO ratio will be much higher than 2, so there will be a need to do something with excess H₂. By the present invention, it is possible to use the total amount of the produced hydrogen in the preparation of the synthesis gas.

In an embodiment according to the first aspect of the invention, the step of providing a carbon dioxide-rich stream and passing it through an electrolysis unit for producing a feed stream comprising CO and CO₂, and the step of providing a water feedstock and passing it through an electrolysis unit for producing a feed stream comprising H₂, are conducted separately, i.e. each step is conducted with its corresponding electrolysis unit.

A higher efficiency when converting the synthesis gas into methanol is achieved: when conducting co-electrolysis there will be some formation of methane as hydrogen and carbon monoxide may react; for methanol production, methane is an inert so there is an efficiency loss associated with the generation of methane.

In addition, by conducting the electrolysis of carbon dioxide and electrolysis of water separately, it is easier to optimize the SOEC stacks of the corresponding electrolysis units and the process for the two different productions. All this while again, the risk of carbon formation is mitigated by not having a full conversion of CO₂, i.e. by operating with partial conversion in the once-through SOEC-CO₂, as explained before.

The electrolysis of CO₂ to CO normally consists of five sections in order to produce high purity CO, for instance 99.9995% CO, namely: feed system, electrolysis, compression, purification e.g in a Pressure Swing Adsorber (PSA) incl. recycle compression, polishing.

When producing methanol, if one was to produce methanol from CO₂ and H₂, this comes at a much higher cost compared to traditional methanol feed gas comprising H₂, CO and CO₂, because the reaction from CO₂ forms water compared to the reaction from CO; again, as a result of the reactions: CO₂+3H₂═CH₃OH+H₂O, CO+2H₂═CH₃OH. The resulting water has a negative effect on the performance of the catalyst and the catalyst volumes increases with more than 100% if the CO₂ concentration is too high, e.g. 90%. Much more energy is also required for the purification of the methanol because all the water is removed by distillation.

The energy to conduct water and carbon dioxide electrolysis is more or less the same, if the energy to evaporate the water is included. Thus, from an energy point of view, generally it does not matter much if one conducts water or carbon dioxide electrolysis where the goal is to produce methanol from water and CO₂.

Normally, a plant or system for conducting CO₂ electrolysis is more complicated (and expensive) than a plant or system for conducting H₂O electrolysis, because it is not possible to have very high conversion of CO₂ in the electrolysis due to carbon formation and because the CO/CO₂ separation is complicated. Therefore, a Pressure Swing Adsorption (PSA) and/recycle compressor-system is required after conducting CO₂ electrolysis. From the PSA a stream rich in CO, normally above 99% CO is withdrawn, as well as a stream rich in CO₂ which is withdrawn at low pressure and therefore is compressed and recycled to the CO₂ electrolysis. However, by conducting a partial conversion, for instance CO/CO₂ of 0.2, 0.25, 0.30, 035, 0.40, 0.45, 0.50, 0.55, 0.6, as recited farther above, the CO₂ electrolysis plant has the same price as a water electrolysis plant per converted molecule. Hence, a simpler and more inexpensive method and plant for producing the synthesis gas is achieved.

By the invention, the electrolysis unit for producing a feed stream comprising CO and CO₂ is a solid oxide electrolysis cell unit, hereinafter also referred to as SOEC-CO₂ (electrolysis of CO₂ via SOEC) is conducted as a once-through operation, i.e. the electrolysis is a once-through electrolysis unit. It would be understood, that the term “conducted” has the same meaning as “operated”. The term “once-through” means that there is no recycling of CO₂ and thereby at least there is no need for a recycle compressor. Compared to traditional systems for conducting CO₂ electrolysis, this embodiment further enables that the need for a recycle compressor is eliminated, and thereby also the need for valves, pipes and control system. Attendant operating expenses such as electric power needed for the compressor as well as maintenance of the recycle compressor and the other equipment (such as valves and pipes), are thereby saved. Moreover, the need for a PSA may also be eliminated, thereby significantly simplifying the process and plant for producing the synthesis gas for further conversion to methanol.

In an embodiment according to the first aspect of the invention, the method comprises by-passing a portion of said a carbon dioxide-rich stream prior to passing it through said solid oxide electrolysis unit. Thereby, increased flexibility in the tailoring of the molar ratio CO/CO₂ in the feed stream comprising CO and CO₂ is possible, while at the same time enabling a smaller solid oxide electrolysis cell unit compared to where no by-pass is provided. For instance, the by-passed portion of the carbon dioxide-rich stream (the feed to the electrolysis unit) mainly containing CO₂ is combined with a stream exiting the electrolysis unit containing CO and CO₂ for thereby producing said feed stream comprising CO and CO₂ having a molar ratio CO/CO₂ of 0.2-0.6.

In an embodiment according to the first aspect of the invention, the synthesis gas has module M=(H₂—CO₂)/(CO+CO₂) or a H₂/CO molar ratio of 1.8-2.1 or 1.9-2.1, preferably 2.

The synthesis gas used for methanol production is normally described in terms of said module M, since the synthesis gas is in balance for the methanol reaction when M=2. In typical synthesis gases for methanol production, such as synthesis gas produced by steam reforming, the synthesis gas will contain some excess hydrogen resulting in modules slightly above 2, for instance 2.05 or 2.1.

In the production of synthesis gas for the further conversion into hydrocarbon products, in particular synthetic hydrocarbon products such as diesel, kerosene, jet fuel, naphtha, it is normal to first prepare a synthesis gas by autothermal reforming (ATR) of a hydrocarbon feed gas, optionally a pre-reformed hydrocarbon feed gas. The hydrocarbon feed gas is typically natural gas. This process scheme for the preparation of synthesis gas is normally referred to as a stand-alone ATR. An Air Separation Unit (ASU) is also needed to supply an oxygen containing stream to the ATR. The thus produced synthesis gas is then passed through a synthetic fuel synthesis unit, from which the above hydrocarbons products are obtained, as well as a tail gas. The synthetic fuel synthesis unit includes typically Fischer-Tropsch (FT) synthesis, from which the tail gas is produced.

Normally the FT synthesis requires a synthesis gas with an H₂/CO-molar ratio of about 2, for example between 1.8 and 2.1. If the hydrocarbon feed to the ATR is natural gas or pre-reformed natural gas, steam and oxygen, the H₂/CO-ratio will typically be higher, such as 2.2-2.4 depending upon a number of factors such as the operating conditions and the natural gas composition. In order to adjust the H₂/CO-ratio to the desired value of ca. 2 as indicated above, it is known to recycle to the ATR part of the tail gas produced in the FT-synthesis.

The present invention provides in contrast to the above conventional methods, a significantly simpler approach for forming a more reactive synthesis gas by tailoring the gas to the desired value of module M for methanol production, or the desired value of H₂/CO molar ratio for FT; in both instances, a value of about 2. Thereby, the size of the corresponding conversion unit, such as the size of the methanol synthesis reactor (methanol reactor) is reduced significantly. In addition, significant savings in electrolysis power consumption is achieved.

The method of the present invention is preferably absent of steam reforming of a hydrocarbon feed gas such as natural gas for producing the synthesis gas. Steam reforming, e.g. conventional steam methane reforming (SMR) or ATR are large and energy intensive processes, hence operation without steam reforming for producing the synthesis gas enables significant reduction in plant size and operating costs as well as significant energy savings. In addition, compared to SMR, with electrolysis units the production capacity can easily be altered by removing or adding more electrolysis units (linear scaling of costs with size). This is normally not the case for e.g. SMR.

The method of the present invention obviates also the use of reverse water gas shift, which can be an expensive and complex solution. Hence, the present invention enables a much simpler method of producing synthesis gas, e.g. for FT-synthesis.

There is a risk for undesired carbon formation in the feed stream comprising CO and CO₂, which may be have a significant content of CO, due to the cooling down of this stream. Hence, in an embodiment according to the first aspect of the invention, the method comprises cooling down said synthesis gas resulting from combining said feed stream comprising CO and CO₂ and said feed stream comprising H₂. In other words, the streams, i.e. the stream comprising CO and CO₂ and the feed stream comprising H₂ and which may also comprise water, for instance up to 25% water are combined before being cooled down. Suitably, said cooling down is from 800 to 400′C. Thereby, the risk of potential carbon formation in the feed stream comprising CO and CO₂ when compressing or went entering other downstream equipment such as a heat exchanger, is reduced or avoided. In particular, metal dusting which is a catastrophic corrosion form which takes place when metals are exposed to CO-rich gas environments, is reduced or avoided.

In an embodiment according to the first aspect of the invention, the step of combining said feed stream comprising CO and CO₂ and said feed stream comprising H₂, is conducted after compressing either stream. In a particular embodiment the synthesis gas from the thus combined streams is subjected to a final compression. For instance, each stream is compressed separately and then combined into the synthesis gas stream having the relevant pressure for the subsequent conversion to methanol or hydrocarbon product, as is well-known in the art. As an example, the feed stream comprising H₂ is made at 20 bar and thereby the feed stream comprising CO and CO₂ must be compressed to 20 bar and then combined into the synthesis gas for final compression. By the invention, whereby partial conversion of the carbon dioxide rich stream is conducted in the once-through SOEC-CO₂, there is also the associated benefit that there is no need for cleaning CO₂ prior to the downstream methanol synthesis.

In some instances, however, cleaning of the carbon dioxide-rich stream prior to electrolysis may be desirable. Accordingly, in an embodiment according to the first aspect of the invention, the carbon dioxide-rich stream is produced by passing a carbon dioxide-feed stream through a CO₂-cleaning unit for removing impurities, such as Cl (e.g. HCl), sulfur (e.g. SO₂, H₂S, COS), Si (e.g. siloxanes), As. This ensures the protection of downstream units, in particular the subsequent electrolysis. For instance, COS even in small amounts can cause problems. Normally, the amount of COS in industrial CO₂ is below the detection limit, but—in certain instances—COS has been measured in the range 10-20 ppb, which is enough to exert a detrimental effect on the electrolysis unit, resulting in a fast degeneration thereof.

In an embodiment according to the first aspect of the invention, the electrolysis unit for producing the feed stream comprising H₂ is an alkaline/polymer electrolyte membrane electrolysis unit i.e. alkaline/PEM electrolysis unit (alkaline cells or polymer cells units).

For the purposes of the present invention, the term alkaline/PEM electrolysis unit means alkaline and/or PEM electrolysis unit.

The combination of using electrolysis of CO₂ via SOEC and electrolysis of water via alkaline/PEM electrolysis further results in electrolysis power reduction compared to the prior art only using electrolysis of water via alkaline/PEM electrolysis with no electrolysis of CO₂.

Furthermore, when the electrolysis of H₂O to H₂ is based on liquid water (like alkaline/PEM), the heat of evaporation of the water is saved.

SOEC-CO₂ and alkaline/PEM electrolysis units are well known in the art, in particular alkaline/PEM electrolysis. For instance, applicant's WO 2013/131778 describes SOECCO₂. The particular combination of SOEC-CO₂ and alkaline/PEM electrolysis is easily accessible and thereby also more inexpensive than other combinations of electrolysis units.

Particularly, in the SOEC-CO₂, CO₂ is converted to a mixture of CO and CO₂ at the fuel electrode i.e. cathode. Also, oxygen is formed at the same time at the oxygen electrode, i.e. anode, often using air as flushing gas. Thus, CO and O₂ are formed on each side of the electrolysis cell.

The present invention enables converting one mole of CO₂ to CO, thereby reducing the need for H₂ for the conversion to methanol by up to one mole, in line with the above reactions for producing methanol, which for the sake of completeness are hereby recited again: CO+2 H₂═CH₃OH; CO₂+3 H₂═CH₃OH+H₂O.

Thus, every time one mole of CO₂ is converted to one mole CO, one mole of H₂ less is needed. This conveys a significant saving in hydrogen consumption.

In an embodiment according to the first aspect of the invention, the electrolysis unit for producing the feed stream comprising H₂ is a solid oxide electrolysis cell unit. Accordingly, both electrolysis units are solid oxide electrolysis cell units (SOEC units). Either of these electrolysis units operates suitably in the temperature range 700-800° C., which thereby enables operating with a common system for the cooling of streams thereof and thus integration of process units. Furthermore, when using SOEC both for electrolysis of CO₂ and for electrolysis of H₂O into H₂ based on steam, the energy for distillation of H₂O out of the produced CH₃OH is saved.

Operation with SOEC units at such high temperatures (700-800° C.) provides advantages over alkaline/PEM electrolysis, which operate at much lower temperature, i.e. in the range 60-160° C. Such advantages include, for instance in connection with CO₂ electrolysis, lower operational expenses due to lower cell voltage as well as lower capital expenses to higher current densities.

In an embodiment according to the first aspect of the invention, said water feedstock comprises steam, or said water feedstock is steam, such as steam produced from other processes of the method, such as from steam generation or downstream distillation. It will be understood, that the term water feedstock includes water (liquid water) and/or steam. Energy efficiency of the process (method) is thereby increased since any steam generated during e.g. downstream process may be reused instead of e.g. requiring steam-export. Also, in the enrichment or purification of e.g. methanol by distillation, water is also formed which advantageously can be reused as part of the water feedstock.

It would be understood, that liquid water cannot be passed through an SOEC, while steam cannot be passed through an alkaline/PEM.

It would also be understood that there will be an overall saving when using water (steam) SOEC for producing H₂ if excess steam is available. Then the evaporation energy is saved in a SOEC which not will be the case if the excess steam is used for power production where the condensation heat will be lost. In particular, there will be excess steam available in the case where the end product is raw methanol, for instance where the raw methanol is produced according to Applicant's U.S. Pat. No. 4,520,216 i.e. methanol-to-gasoline route (TiGAS), where raw methanol is converted to gasoline, or if the synthesis gas is used for substitute natural gas (SNG).

In an embodiment according to the first aspect of the invention, said carbon dioxide-rich stream comprises carbon dioxide from external sources such as from biogas upgrading or fossil fuel-based syngas (synthesis gas) plants.

External sources, as mentioned above, include biogas upgrade. Biogas is a renewable energy source that can be used for heating, electricity, and many other operations. Biogas can be cleaned and upgraded to natural gas standards, when it becomes bio-methane. Biogas is primarily methane (CH₄) and carbon dioxide (CO₂), typically containing 60-70% vol. methane. Up to 30% or even 40% of the biogas may be carbon dioxide. Typically, this carbon dioxide is removed from the biogas and vented to the atmosphere in order to provide a methane rich gas for further processing or to provide it to a natural gas network. The removed CO₂ is utilized for making more syngas with the method according to the present invention.

An example of a fossil fuel-based syngas plant is a natural gas-based syngas plant for FT or for gasoline production (TiGAS) i.e. a Gas-to-Liquid (GTL) process, or for methanol production where CO₂ is extracted from waste heat sections or fired heater flue gases and utilized for making more syngas with the method according to the present invention.

Other external sources include heat and power plants and waste incineration plants.

In an embodiment according to the first aspect of the invention, the electrical power required in the step of electrolysis of the carbon dioxide-rich stream or the water feedstock, is provided at least partly by renewable sources, such as wind and solar energy, or for instance also by hydropower. Thereby an even more sustainable i.e. “greener” method (process) and system (plant) approach is achievable, since no fossil fuels are used for the generation of power needed for the electrolysis.

In an embodiment according to the first aspect of the invention, the step of converting the synthesis gas into methanol comprises passing the synthesis gas through a methanol synthesis reactor under the presence of a catalyst for producing a raw methanol stream, said step optionally further comprising a distillation step of the raw methanol stream for producing a water stream and a separate methanol stream having at least 98 wt % methanol. The molar ratio of CH₃OH/H₂O in the raw methanol stream according to the present invention is 1.2 or higher, for instance 1.3 or higher. Thus, the synthesis gas is more reactive than in conventional methanol synthesis or where only water electrolysis is used for producing hydrogen. In conventional methanol synthesis, from the so-called methanol loop a raw methanol product is produced having a molar ratio CHOH/H₂O of often about 1, which represents the production of a substantial amount of water which needs to be separated downstream. Hence, the present invention further enables that the produced raw methanol has a much lower content of water, e.g. at least 20% or at least 30% less water on a molar base, compared to conventional methanol synthesis, thereby enabling less water being carried on in the process with attendant reduction in e.g. equipment size, such as piping, as well as reducing the costs of water separation downstream, e.g. by enabling a much simpler and cost efficient distillation for the purification of methanol. Furthermore, the catalyst performance in the methanol synthesis reactor is also sensitive to water, so catalyst volumes and thereby reactor size are further reduced.

Methanol technology including methanol synthesis reactors and/or methanol synthesis loops are well-known in the art. Thus, the general practice in the art is conducting the methanol conversion in a once-through methanol conversion process; or to recycle unconverted synthesis gas separated from the reaction effluent and dilute the fresh synthesis gas with the recycle gas. The latter typically results in the so-called methanol synthesis loop with one or more reactors connected in series or in parallel. For instance, serial synthesis of methanol is disclosed in U.S. Pat. Nos. 5,827,901 and 6,433,029, and parallel synthesis in U.S. Pat. No. 5,631,302 and EP 2874738 B1.

In a second aspect of the invention, there is provided a method for producing a hydrocarbon product such as a synthetic fuel, comprising the steps of:
providing a carbon dioxide-rich stream and passing it through an electrolysis unit for producing a feed stream comprising CO and CO₂,
providing a water feedstock and passing it through an electrolysis unit for producing a feed stream comprising H₂,
combining said feed stream comprising CO and CO₂ and said feed stream comprising H₂ into a synthesis gas,
converting said synthesis gas into said hydrocarbon product,
   wherein the step of providing a carbon dioxide-rich stream and passing it through an electrolysis unit for producing a feed stream comprising CO and CO₂, is conducted as a once-through operation in a solid oxide electrolysis cell unit i.e. SOEC-CO₂,
   wherein the feed stream comprising CO and CO₂, or the synthesis gas, has a molar ratio CO/CO₂ of 0.8 or higher such as 0.9, and wherein the step of converting the synthesis gas into a hydrocarbon product comprises passing the synthesis gas through a Fischer-Tropsch (FT) synthesis unit.

In an embodiment, tail gas (FT-tail gas) is produced from the FT synthesis unit. The tail gas may be used for providing said carbon dioxide-rich stream, as recited below. In another embodiment, the synthetic fuel is any of diesel, kerosene, jet fuel, naphtha, in particular diesel.

As for methanol technology, FT technology is also well-known in the art and reference is particularly given to Steynberg A. and Dry M. “Fischer-Tropsch Technology”, Studies in Surface Sciences and Catalysts, vol. 152.

In an embodiment, said carbon dioxide-rich stream comprises carbon dioxide produced from said tail gas, i.e. FT-tail gas, produced in the step of converting the synthesis gas to said hydrocarbon product. The recycle of FT-tail gas, which is normally CO₂-rich, is highly advantageous since otherwise the tail gas will need to be exported as a fuel source, given that FT-tail gas also normally contains methane and a lesser extent of other hydrocarbons.

In a third aspect, the invention encompasses also a system, i.e. a plant or process plant, for producing methanol or a hydrocarbon product such as a synthetic fuel, comprising:
a once-through solid oxide electrolysis cell unit, arranged to receive a carbon dioxide-rich stream for producing a feed stream comprising CO and CO₂ and to produce a feed stream comprising CO and CO₂,
an electrolysis unit arranged to receive a water feedstock for producing a feed stream comprising H₂,
a compressor section arranged to receive the feed stream comprising CO and CO₂ and the feed stream comprising H₂, for compressing and combining said streams into a synthesis gas,
a methanol synthesis unit arranged to receive said synthesis gas for producing said methanol, preferably having a concentration, i.e. purity, of at least 98% methanol, wherein said once-through solid oxide electrolysis unit is arranged to produce said feed stream comprising CO and CO₂ or the synthesis gas, with a molar ratio CO/CO₂ of 0.2-0.6; or
a hydrocarbon product synthesis unit, preferably a Fischer-Tropsch (FT) synthesis unit, for producing said hydrocarbon product, such as a synthetic fuel, e.g. diesel, wherein said once-through solid oxide electrolysis unit is arranged to produce said feed stream comprising CO and CO₂, or the synthesis gas, with a molar ratio CO/CO₂ of 0.8 or higher, such as 0.9.

As with the method according to the first aspect of the invention, a more reactive synthesis gas is formed thereby enabling a smaller size of the downstream rector such as a methanol synthesis reactor, there will be less formation of water in e.g. the methanol synthesis loop and thereby equipment size is reduced as so is the cost of water separation. By less water formation, catalyst volume and thereby the size of the methanol synthesis unit is further reduced. Furthermore, as with the method according to the first aspect of the invention, the system enables converting one mole of CO₂ to CO, thereby reducing the need for H₂ by up to one mole for every mole of methanol produced.

Any of the embodiments and associated benefits of the first or second aspect of the invention may be used together with any of the embodiments of the third aspect of the invention, or vice versa.

### BRIEF DESCRIPTION OF THE DRAWINGS

FIG. 1 shows a schematic method and system (process and plant) for the production of a synthesis gas for further conversion to methanol according to the prior art.

FIG. 2 shows a schematic method and system for the production of synthesis gas and further conversion to methanol according to an embodiment of the invention.

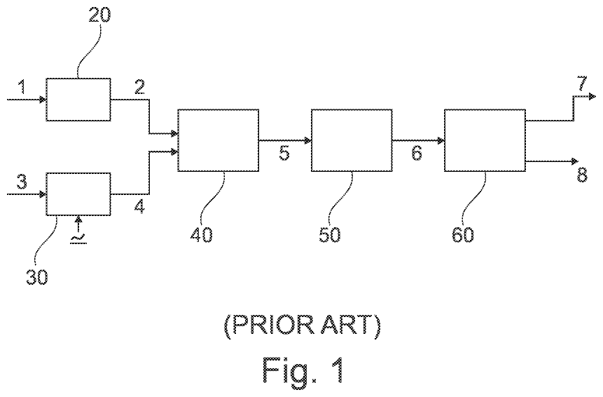

With reference to FIG. 1 (prior art), a carbon dioxide-feed stream 1 is passed through a CO₂-cleaning unit 20 for removing impurities and thereby producing a CO₂-rich stream 2. A water feedstock 3 passes through an electrolysis unit 30 such as an alkaline/PEM-electrolysis unit powered by a sustainable source such as wind or solar energy, thereby producing a feed stream 4 comprising H₂, i.e. a H₂-rich stream. Both streams 2 and 4 pass through a compression section 40 whereby they are compressed and combined into a synthesis gas stream 5 having a molar ratio H₂/CO₂ of about 3. While the module “M” defined previously is used in any gas mixture comprising carbon dioxide and carbon monoxide and hydrogen, the molar ratio of hydrogen to carbon dioxide is only relevant to use for a gas mixture of carbon dioxide and hydrogen. The synthesis gas 5 enters the methanol loop 50 as is well-known in the art, whereby the synthesis gas 5 is converted to a raw methanol stream 6 having a molar ratio CH₃OH/H₂O of about 1. The water in the raw methanol stream 6 is then removed in distillation unit 60, where the raw methanol stream 6 is then purified or enriched in methanol. A methanol product 7 having a concentration of at least 98 wt % is then produced, as well as water stream 8.

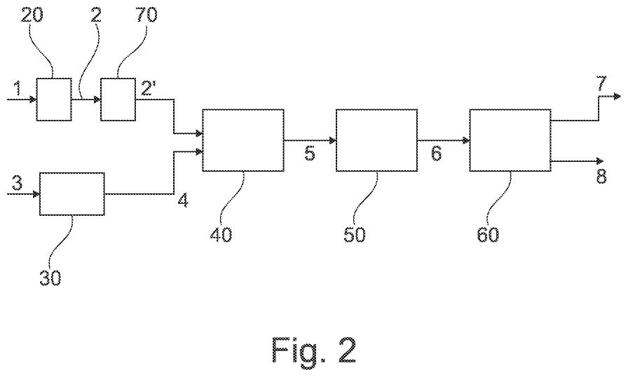

Now with reference to FIG. 2, which is in accordance with an embodiment of the invention, a carbon dioxide-feed stream 1 is passed through a CO₂-cleaning unit 20 for removing impurities and producing a CO₂-rich stream 2, and then through an electrolysis unit 70, here a once-through SOEC-CO₂ unit, which is also powered by a sustainable source such as wind or solar energy, thereby producing a feed stream 2′ comprising CO and CO₂ and having a molar ratio CO/CO₂ above 0.2, in particular 0.2-0.6. Separately, the water feedstock 3 also passes through an electrolysis unit 30, such as a PEM-electrolysis unit or SOEC unit also powered by a sustainable source, thereby producing a feed stream 4 comprising H₂. Both streams 2′ and 4 pass through a compression section 40 whereby they are compressed and combined into a now more reactive synthesis gas stream 5 having a module M=(H₂—CO₂)/(CO+CO₂) which is highly suitable for the downstream conversion into methanol. This synthesis gas 5 enters the methanol loop 50 as is well-known in the art, whereby it is converted to a raw methanol stream 6 now having a molar ratio CH₃OH/H₂O of 1.3 or higher, i.e. at least 30% less water on a molar basis compared to the prior art. The water in the raw methanol stream 6 is then more expediently removed in distillation unit 60, where this stream is purified or enriched in methanol. A methanol product 7 having a concentration of at least 98 wt % is then produced, as well as water stream 8 which may be used as part of the water feedstock 3.

### EXAMPLE

The results of below Table 1 correspond to a plant for producing methanol for 100 kmol/h CO₂ with water (steam) electrolysis (SOEC) only for producing H₂ (prior art) in accordance with the reaction: 3 H₂+CO₂═CH₃OH+H₂O; and with water (steam) electrolysis (SOEC) for producing H₂ and CO₂ electrolysis (SOEC-CO₂) for producing CO (invention) in accordance with the reaction: CO+2 H₂═CH₃OH:

TABLE 1

| | | Invention | | |
|---|---|---|---|---|
| | Prior art | H₂O + | | |
| | H₂O | 100% CO₂ | | |
| | electrolysis | electrolysis, | Improve- | |
| | only, MW | MW | ment | Comments |
| H₂O electrolysis | 23.85 | 15.90 | | 100% |
| (SOEC) | | | | efficiency |
| CO₂ electrolysis | | 7.86 | | 100% |
| (SOEC) | | | | efficiency |
| Sum electrolysis | 23.85 | 23.76 | 0.4% | |
| Compression to | 2.68 | 2.18 | 18.7% | 14% work |
| 90 bar g | | | | loss |
| Duty for steam | 1.45 | 2.46 | −69.4% | 0% heat |
| generation | | | | loss/25 |
| | | | | C. temp. |
| | | | | approach in |
| | | | | heat exch. |
| Duty for air and | 2.13 | 1.08 | 49.4% | |
| water coolers | | | | |

Thus, there is 19% saving for the compressor power due to lower gas volume and density; 70% more duty for steam generation—and corresponding 50% less heat lost in coolers. Thus, with the same efficiency by using SOEC for both H₂O-electrolysis and CO₂-electrolysis, there will be no significant savings in electrolysis power. However, by operating SOEC for both H₂O-electrolysis and CO₂-electrolysis in accordance with the invention enables operating with a common system for the cooling of streams thereof, as both SOEC units operate in the same temperature range of about 700-800′C, and thus better integration of process units. Further, as SOEC utilizes steam, the energy for distillation of H₂O out of the produced methanol is saved.

Table 2 below compares now the prior art with water (liquid) electrolysis only (alkaline/PEM electrolysis) for producing H₂ in accordance with the reaction: 3 H₂+CO₂═CH₃OH+H₂O; and an embodiment of the invention with water (liquid) electrolysis (alkaline/PEM electrolysis) for producing H₂ as well as CO₂ electrolysis (SOEC-CO₂) for producing CO in accordance with the reaction: CO+2 H₂═CH₃OH:

TABLE 2

| | | Invention | | |
|---|---|---|---|---|
| | Prior art | H₂O + | | |
| | H₂O | 100% CO₂ | | |
| | electrolysis | electrolysis, | Improve- | |
| | only, MW | MW | ment | Comments |
| H₂O electrolysis | 29.81 | 19.87 | | 80% |
| (alkaline/PEM) | | | | efficiency |
| CO₂ electrolysis | | 7.86 | | 100% |
| (SOEC) | | | | efficiency |
| Sum electrolysis | 29.81 | 27.74 | 7.0% | |

Thus, when using alkaline/PEM for H₂O-electrolysis and SOEC for CO₂-electrolysis in accordance to an embodiment of the invention, there is 7% reduction (improvement) in power consumption with respect to only using alkaline/PEM for producing H₂. The invention according to this embodiment enables therefore not only the formation of a more reactive synthesis gas, but also a reduction in electrolysis power consumption.

1. A method for producing methanol, comprising the steps of:
providing a carbon dioxide-rich stream and passing it through an electrolysis unit for producing a feed stream comprising CO and CO₂
providing a water feedstock and passing it through an electrolysis unit for producing a feed stream comprising H₂,
combining said feed stream comprising CO and CO₂ and said feed stream comprising H₂ into a synthesis gas,
converting said synthesis gas into said methanol,
wherein the step of providing a carbon dioxide-rich stream and passing it through an electrolysis unit for producing a feed stream comprising CO and CO₂, is conducted as a once-through operation in a solid oxide electrolysis cell unit, and
wherein the molar ratio CO/CO₂ in the feed stream comprising CO and CO₂, or the synthesis gas, is in the range 0.2-0.6.

2. Method according to claim 1, wherein the molar ratio CO/CO₂ in the feed stream comprising CO and CO₂, or the synthesis gas, is 0.25, 0.30 or 0.35, 0.40 or 0.45, 0.50 or 0.55.

3. Method according to claim 1, wherein the step of providing a carbon dioxide-rich stream and passing it through an electrolysis unit for producing a feed stream comprising CO and CO₂, and the step of providing a water feedstock and passing it through an electrolysis unit for producing a feed stream comprising H₂, are conducted separately.

4. Method according to claim 1, comprising by-passing a portion of said a carbon dioxide-rich stream prior to passing it through said solid oxide electrolysis unit.

5. Method according to claim 1, comprising cooling down said synthesis gas resulting from combining said feed stream comprising CO and CO₂ and said feed stream comprising H₂, suitably cooling down from 800-400′C.

6. Method according to claim 1, wherein the step of combining said feed stream comprising CO and CO₂ and said feed stream comprising H₂, is conducted after compressing either stream.

7. Method according to claim 1, wherein the carbon dioxide-rich stream is produced by passing a carbon dioxide-feed stream through a CO₂ cleaning unit for removing impurities, such as Cl, sulfur, Si, As.

8. Method according to claim 1, wherein the electrolysis unit for producing the feed stream comprising H₂ is an alkaline/polymer electrolyte membrane electrolysis unit i.e. alkaline and/or PEM electrolysis unit.

9. Method according to claim 1, wherein the electrolysis unit for producing the feed stream comprising H₂ is a solid oxide electrolysis cell unit.

10. Method according to claim 1, wherein said water feedstock comprises steam such as steam produced from other processes of the method, such as from steam generation or downstream distillation.

11. Method according to claim 1, wherein said carbon dioxide-rich stream comprises carbon dioxide from external sources such as from biogas upgrading or fossil fuel-based syngas plants.

12. The method according to claim 1, wherein the electric power required in the step of electrolysis of the carbon dioxide-rich stream or the water feedstock, is provided at least partly by renewable sources, such as wind and solar energy.

13. Method according to claim 1, wherein the step of converting the synthesis gas into methanol comprises passing the synthesis gas through a methanol synthesis reactor under the presence of a catalyst for producing a raw methanol stream, said step optionally further comprising a distillation step of the raw methanol stream for producing a water stream and a separate methanol stream having at least 98 wt % methanol.

14. A method for producing a hydrocarbon product such as a synthetic fuel, comprising the steps of:
providing a carbon dioxide-rich stream and passing it through an electrolysis unit for producing a feed stream comprising CO and CO₂,
providing a water feedstock and passing it through an electrolysis unit for producing a feed stream comprising H₂,
combining said feed stream comprising CO and CO₂ and said feed stream comprising H₂ into a synthesis gas,
converting said synthesis gas into said hydrocarbon product,
wherein the step of providing a carbon dioxide-rich stream and passing it through an electrolysis unit for producing a feed stream comprising CO and CO₂, is conducted as a once-through operation in a solid oxide electrolysis cell unit,
wherein the feed stream comprising CO and CO₂, or the synthesis gas, has a molar ratio CO/CO₂ of 0.8 or higher such as 0.9, and
wherein the step of converting the synthesis gas into a hydrocarbon product comprises passing the synthesis gas through a Fischer-Tropsch (FT) synthesis unit.

15. A system for the production of methanol or a hydrocarbon product such as a synthetic fuel, comprising:
a once-through solid oxide electrolysis cell unit, arranged to receive a carbon dioxide-rich stream for producing a feed stream comprising CO and CO₂ and to produce a feed stream comprising CO and CO₂,
an electrolysis unit arranged to receive a water feedstock for producing a feed stream comprising H₂,
a compressor section arranged to receive the feed stream comprising CO and CO₂ and the feed stream comprising H₂, for compressing and combining said streams into a synthesis gas,
a methanol synthesis unit arranged to receive said synthesis gas for producing said methanol, preferably having a concentration, i.e. purity, of at least 98% methanol, wherein said once-through solid oxide electrolysis unit is arranged to produce said feed stream comprising CO and CO₂ or the synthesis gas, with a molar ratio CO/CO₂ of 0.2-0.6; or
a hydrocarbon product synthesis unit, preferably a Fischer-Tropsch (FT) synthesis unit, for producing said hydrocarbon product, such as a synthetic fuel, wherein said once-through solid oxide electrolysis unit is arranged to produce said feed stream comprising CO and CO₂, or the synthesis gas, with a molar ratio CO/CO₂ of 0.8 or higher, such as 0.9.
